(19) United States
(12) Patent Application Publication
(10) Pub. No.: US 2015/0038973 A1
(43) Pub. Date: Feb. 5, 2015

(54) Methods and Devices for Implementing an Improved Rongeur

(71) Applicant: OsteoMed LLC, Addison, TX (US)

(72) Inventor: Gregory Andrew Grim, Lascassas, TN (US)

(73) Assignee: OsteoMed LLC, Addison, TX (US)

(21) Appl. No.: 14/447,418

(22) Filed: Jul. 30, 2014

Related U.S. Application Data
(60) Provisional application No. 61/861,725, filed on Aug. 2, 2013.

(51) Int. Cl. A61B 17/16

(52) U.S. Cl. CPC A61B 17/1611
USPC 606/83

(57) ABSTRACT

A rongeur which functions to act on (or bite) bone while approaching the bone surface from an axial orientation is provided. In one embodiment, a rongeur is provided with a rotatably/pivotally coupled cutting jaw component to cause the cutting jaw to exert force on the bone. The rongeur may include an axially sliding component which provides axial force and actuates a cutting jaw such that it pivots toward a bone surface. Additionally, a lip feature which creates an opposing surface wherein the cutting jaw pivots toward that surface in order to bite the bone may also be provided. The lip feature may act as a shim between dura mater tissue and the inferior surface of a bone. An axial rongeur may be configured to bite and remove bone when placed while under the surface of a patient's skin (e.g. after axially traversing a distance under the skin).

### CROSS-REFERENCE TO RELATED APPLICATION

The present application claims priority to co-pending U.S. Provisional Patent Application No. 61/861,725, entitled “METHODS AND DEVICES FOR IMPLEMENTING AN IMPROVED RONGEUR,” filed Aug. 2, 2013, the disclosure of which is hereby incorporated herein by reference in its entirety.

### TECHNICAL FIELD

The present disclosure relates generally to a rongeur which functions to act on (or bite) bone while approaching the bone surface from an axial orientation.

### BACKGROUND

Craniosynostosis is a condition in which one or more of the fibrous sutures in an infant skull prematurely fuses by turning into bone (ossification), thereby changing the growth pattern of the skull. When such a condition occurs, because the skull cannot expand perpendicular to the fused suture, it may compensate by growing more in the direction parallel to the closed sutures. Sometimes the resulting growth pattern provides the necessary space for the growing brain, but it may result in an abnormal head shape and abnormal facial features. In cases in which the compensation does not effectively provide enough space for the growing brain, craniosynostosis may result in increased intracranial pressure leading possibly to visual impairment, sleeping impairment, eating difficulties, or an impairment of mental development combined with a significant reduction in IQ.

Many methods for treating craniosynostosis have been implemented. One relatively new method that is gaining acceptance entails creating a strip craniectomy and inserting a device configured to maintain separation of the newly-defined areas of the skull. In such a procedure, a portion of the skull (e.g. a 1 cm wide section) is removed and spring is placed into the void generated by the removal of the skull portion, which then exerts outward force on the skull from within the removed portion in order to facilitate the proper, more anatomical, growth vector in the skull.

While such a treatment has shown to be a marked improvement over earlier methods, some issues may still arise which are associated with this procedure. For example, a strip craniectomy may be invasive to the patient because of the space required (and corresponding length of incision) to remove the bone, etc. It is appreciated that there are a number of additional problems which are associated with higher invasiveness of a surgery. For example, additional complexity of a surgery may result in longer anesthesia times for the patient undergoing the surgery. Increased anesthesia doses and time may increase possible adverse effects resulting from the anesthesia drugs. Further, longer surgery times and larger incisions may cause additional blood loss to occur, as well as increase the time required for patient healing. Generally larger incisions are currently required because the current surgical instruments being employed are general designs and not specifically suited for this procedure.

One instrument that is utilized to remove prematurely fused bone is a Kerrison-type rongeur. FIG. 1 illustrates a typical Kerrison-type rongeur 100 and typical bite orientation options 101-103 currently available. Rongeur 100 is configured to perform cutting in a plane that is substantially perpendicular to the bone structure being removed. Accordingly, to remove a strip of bone from a skull, rongeur 100 of FIG. 1 must be oriented perpendicularly with respect to the skull and bite the bone along a surface plane of the skull. Therefore, the corresponding incision on the scalp must be large enough (e.g. at least the length of the strip to be removed) to accommodate the use of this tool.

### BRIEF SUMMARY

The present application provides for systems, methods and devices which provide for a rongeur which functions to act on (or bite) bone while approaching the bone surface from an axial orientation. In one embodiment, a rongeur is provided with a rotatably/pivotally coupled cutting jaw component to cause the cutting jaw to exert force on the bone. In some embodiments, the rongeur includes an axially sliding component which provides axial force and actuates the cutting jaw such that it pivots toward a bone surface. Such an axial force may be provided by a user actuating a lever which transfers the force to the sliding component or by other means (e.g. electronic/automatic and the like). Additionally, in some embodiments a lip feature, which creates an opposing surface, wherein the cutting jaw pivots toward that surface in order to bite the bone may also be provided. The lip feature may also act as a shim between dura mater tissue and the inferior surface of a bone. In yet another embodiment, an axial rongeur may be configured such that it may bite and remove bone when placed while under the surface of a patient's skin (e.g. after axially traversing a distance under the skin from an opening/point of incision). In some embodiments, the lip feature may be configured to define a reservoir for collecting excised bone.

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 illustrates an exemplary perpendicular-type rongeur and typical bite orientation options available;

FIG. 2 illustrates an axial rongeur in accordance with an embodiment of the present application; and

FIG. 3 illustrates an exploded view of the end portion of the rongeur shown in FIG. 2.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

As an initial matter, it is noted that while some of the discussion herein is provided in the context of performing a craniectomy, it is appreciated that embodiments may be functional when used in other portions of the body. Accordingly, such a discussion is provided for illustrative purposes in order to highlight advantages of the embodiments of the present disclosure.

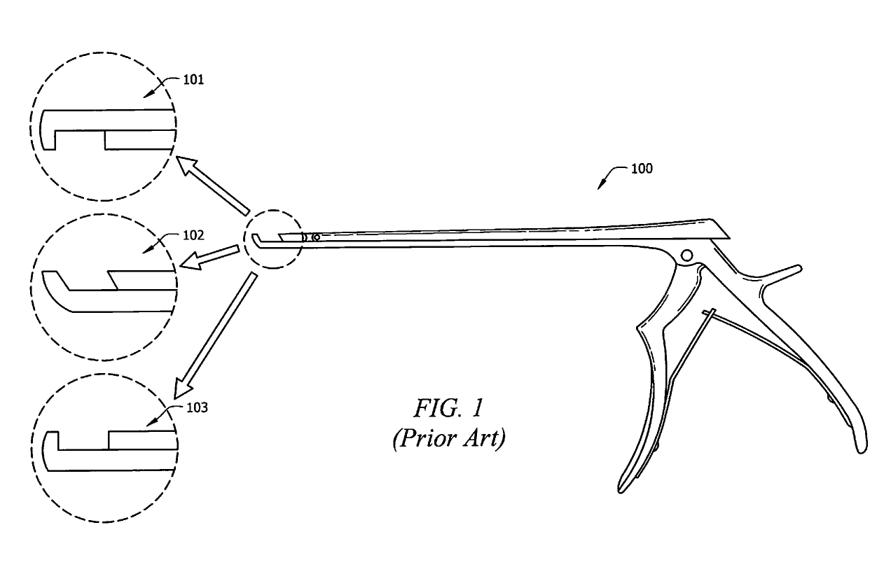

Additionally, the term “axial” is used to describe the approach of devices with respect to a bone surface in accordance with embodiments of the present application. It is appreciated that one of skill in the art would understand that the term axial may refer to a substantially axial approach as the subject bone will not always reside on a precise axis and some degree of elevated angle of approach may be needed to allow a portion of the rongeur (e.g. a lip portion) to contact the inferior side of a bone surface. For example, in some embodiments, an angle as great as 45 degrees with respect to a bone surface may be considered “axial.” The term axial is meant to distinguish from a perpendicular-type rongeur (shown in FIG. 1). It is noted that an axial rongeur may provide advantages in how the device approaches a target bone surface which are not possible with a perpendicular-type rongeur. Such advantages are discussed below and will be apparent to one of skill in the art reading the present disclosure.

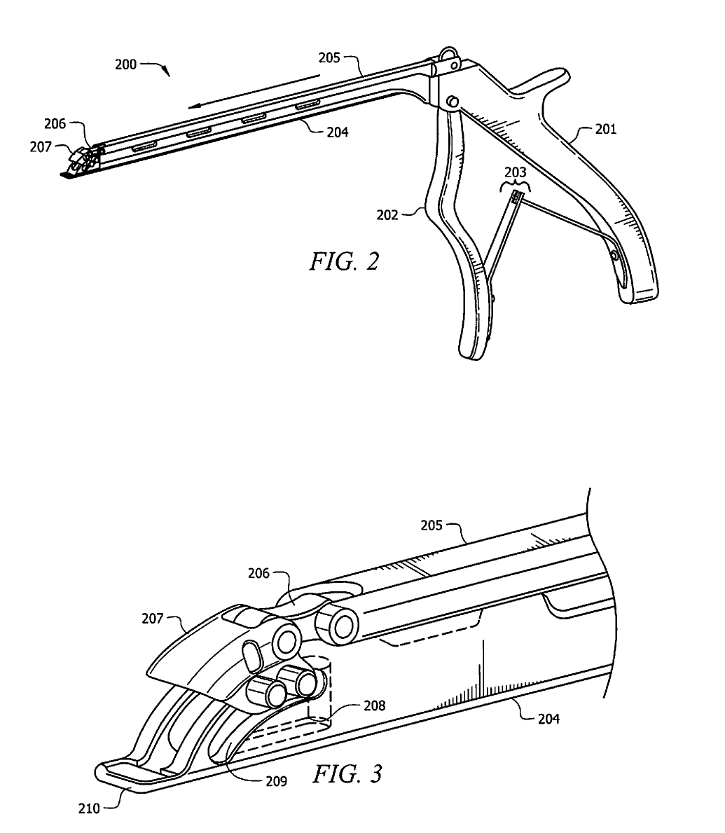

FIG. 2 illustrates an axial rongeur 200 in accordance with an embodiment of the present application. Rongeur 200 includes handle 201, lever component 202 and leafspring components 203 disposed therebetween. Rongeur 200 further includes a body portion 204 and a sliderail component 205 disposed thereon which is slidably coupled to body portion 204. Sliderail component 205 is coupled to lever component 202 in a manner where force exerted on lever component 202 actuates a sliding motion of sliderail component 205 to slide along body portion 204.

Rongeur 200 further includes link component 206 which couples sliderail component 205 to jaw component 207 (see FIG. 3 which illustrates an exploded view of the end portion of rongeur 200). Link component 206 is configured with a pivoting component which allows jaw component 207 to pivotally rotate downward. Jaw component 207 is coupled to link component 206, which is coupled to sliderail component 205 and body portion 204 such that jaw component 207 is configured to pivot/rotate while also moving axially in order to provide a biting force. In the illustrated embodiment, jaw component 207 is coupled to body portion 204 with a plurality of pins 208 which extend through body portion 204 and slide within guide portion 209. This coupling provides a downward directional path of travel jaw component 207 when jaw component 207 receives axial force.

Body portion 204 further comprises lip portion 210. Lip portion 210 serves to provide reactive force against compressive force exerted by jaw component 207. This compressive force may be the result of a perpendicular force exerted by jaw component 207 on lip portion 210 as it pivotally rotates downward, with respect to the axial approach, upon receiving the axial force from sliderail component 205. Lip portion 210 may act as a shim between dura mater tissue and the inferior surface of a bone, while providing the reactive force against the inferior surface of the bone. In this manner, for example, when jaw component 207 rotates downward, with respect to the axial approach, and exerts a compressive force against the superior surface of the bone, the reactive force and the compressive force create a biting force that allows removal of the subject bone. In some embodiments, lip portion 210 may be configured as a secondary cutting edge that mirrors that of jaw component 207. In such embodiments, when downward force is exerted by jaw component 207, the cutting edge of lip portion 210 provides a sharp edge that assists in the cutting of the bone.

In some embodiments, lip portion 210 may be configured to define a reservoir within the body portion 204 for excised bone debris. The reservoir may be configured to collect bone fragments as these fragments are being excised from the subject bone. Such a configuration may prevent (or substantially prevent) bone fragments from being deposited within the body. For example, when performing a craniectomy, collecting excised bone in the reservoir assists to prevent bone fragments from contaminating the surgical site. In one embodiment, the reservoir may be defined and manufactured as part of lip portion 210. In other embodiments, the reservoir may be manufactured separately and lip portion 210 may be configured to couple with the reservoir.

In use, when lever component 202 is actuated (e.g. pulled toward handle 201 by a user), the force is transferred to sliderail component 205 which in turn exerts axial force to jaw component 207 through link component 206. Jaw component 207 is restrained from moving in a purely axial direction with respect to rongeur 200 due to its coupling to body portion 204 via pins 208. Therefore, jaw component 207 moves axially, as it slides within guide portion 209, while also pivoting/rotating perpendicularly with respect to the axis of the axial approach. This perpendicular motion provides downward (or upward) biting force on a subject bone against lip portion 210.

Accordingly, rongeur 200 may axially approach a subject bone structure to be removed. Such a configuration provides multiple advantages. For example, in conducting a strip craniectomy, a surgeon may create a single incision in which the axial rongeur may slide underneath to remove a length of bone material (thereby potentially eliminating the need for the long incision needed for a perpendicular approach). Such an incision may be made, e.g., perpendicular on the surface with respect to the axial approach of the rongeur in order to allow for enough width to fit the rongeur tool underneath the skin surface. Accordingly, treatments for craniosynostosis may be implemented with a single incision surgery where the single incision is minimally invasive with respect to prior methods.

It is noted that the rongeur 200 is one example implementation and there are many ways that the illustrated design could be altered and still be within the scope of the present invention. For example, handle 201 and lever 202 may be implemented different ways to provide for the energy of the system. For example, different handles may be used which accept different types of force/system energy (e.g. rotational, pressure, etc.), motorized embodiments may be employed, and the like. Further, while sliderail component 205 is utilized to provide axial force, different mechanisms may be utilized, e.g. internal rigid components, pulley/spring components, etc. Anything which will allow a force from an axial direction to be provided to jaw component 207 may be utilized. Further, couplings between the jaw component and either the axial force component or body may be implemented in any manner which allows the directional force needed to be accomplished. In other words, it is appreciated that rongeur 200 may be implemented in any manner which allows an axial approach to be translated into a perpendicular-style (e.g. either downward or upward) biting force with respect to the axis of the axial approach in accordance with embodiments of the present invention.

Although the embodiments of the present disclosure and their advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A rongeur configured to axially approach a subject bone structure, said rongeur comprising:
a body component;
a jaw component coupled to the body component at a guide portion;
at least one axial force component, said axial force component configured to be coupled to the jaw component and to provide axial energy to the jaw component; and
wherein, upon the jaw component receiving axial force from the axial force component, the jaw component is configured to move along the guide portion of the body component to provide perpendicular force, with respect to the axial approach, onto the bone structure.

2. The rongeur of claim 1 wherein said jaw component comprises a primary cutting edge.

3. The rongeur of claim 1 further comprising:
a lip portion configured to provide a surface toward which said jaw component moves when receiving said axial force, said surface adapted to provide a reactive force against a compressive force exerted by said jaw component as a result of said perpendicular force.

4. The rongeur of claim 3 wherein said surface is adapted to provide a secondary cutting edge, said secondary cutting edge mirroring said primary cutting edge.

5. The rongeur of claim 3 wherein said lip portion is configured to define a reservoir for excised bone debris.

6. The rongeur of claim 1 wherein said jaw component is coupled to the body component using at least one pin, said at least one pin configured to slide within said guide portion.

7. The rongeur of claim 1 wherein said axial force component comprises a sliderail, said sliderail slidably coupled to said body component and configured to provide said axial force to said jaw component.

8. The rongeur of claim 1 further comprising an electronic actuator, said electronic actuator configured to generate said axial force.

9. The rongeur of claim 8 wherein said electronic actuator is automatically actuated.

10. A method to excise bone from a subject bone structure using a rongeur configured to axially approach said subject bone structure comprising:
providing axial force energy to an axial force component of said rongeur, said axial force component configured to be coupled to a jaw component of said rongeur;
transferring said axial force energy to said jaw component;
moving, in response to said transferring, said jaw component along a guide portion of said rongeur;
providing, in response to said moving, a perpendicular force, with respect to the axial approach, onto said bone structure.

11. The method of claim 10 wherein said jaw component is adapted provide a primary cutting edge.

12. The method of claim 10 further comprising:
providing, by a lip portion of said rongeur, a reactive force against a compressive force exerted by said jaw component as a result of said perpendicular force, said lip portion configured to provide a surface toward which said jaw component moves when said axial force energy is transferred from said axial force component.

13. The method of claim 12 wherein said surface is adapted to provide a secondary cutting edge, said secondary cutting edge mirroring said primary cutting edge.

14. The method of claim 12 further comprising collecting excised bone debris in a reservoir defined by said lip portion.

15. The method of claim 10 wherein said axial force component comprises a sliderail, said sliderail slidably coupled to said rongeur and configured to provide said axial force to said jaw component.

16. The method of claim 10 further comprising generating said axial force using an electronic actuator.

17. The method of claim 16 wherein said electronic actuator is automatically actuated.

18. A method for manufacturing a rongeur configured to axially approach a subject bone structure, the method comprising:
forming a body component;
forming a jaw component coupled to the body component at a guide portion; and
forming at least one axial force component, said axial force component configured to be coupled to the jaw component and to provide axial energy to the jaw component;
wherein, upon the jaw component receiving axial force from the axial force component, the jaw component is configured to move along the guide portion of the body component to exert perpendicular force, with respect to the axial approach, onto the bone structure.

19. The method of claim 18 wherein said jaw component comprises a primary cutting edge.

20. The method of claim 18 further comprising:
forming a lip portion configured to provide a surface toward which said jaw component moves when receiving said axial force, said surface adapted to provide a reactive force against a compressive force exerted by said jaw component as a result of said perpendicular force.

21. The method of claim 20 wherein said surface is adapted to provide a secondary cutting edge, said secondary cutting edge mirroring said primary cutting edge.

22. The rongeur of claim 20 wherein said lip portion is configured to define a reservoir for excised bone debris.

23. The method of claim 18 wherein said jaw component is coupled to the body component using at least one pin, said at least one pin configured to slide within said guide portion.

24. The method of claim 18 wherein said axial force component comprises a sliderail, said sliderail slidably coupled to said body component and configured to provide said axial force to said jaw component.

25. The method of claim 18 further comprising an electronic actuator, said electronic actuator configured to generate said axial force.

26. The method of claim 26 wherein said electronic actuator is automatically actuated.
